SEQUENCE LISTING (104 sequences)

SEQ ID NO: 1 - protein, 130 aa, Murine
SRDIPMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKRADAAPTVSIFPPSSEQLTSG

SEQ ID NO: 2 - protein, 129 aa, Murine
SREIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELKRADAAPTVSIFPPSSEQLTSG

SEQ ID NO: 3 - protein, 134 aa, Murine
SRDNVLTQSPASLAVSPGQRATISCKASQSVDYDGDNYMNWYQQKPGQPPKLLIYAASNLESGIPARFSGSGSGTDFTLNIHPVEEEDAATYFCQQSDEDPYTFGGGTKLEIKRADAAPTVSIFPPSSEQLTSG

SEQ ID NO: 4 - protein, 130 aa, Murine
SRDIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKRADAAPTVSIFPPSSEQLTSG

SEQ ID NO: 5 - protein, 130 aa, Murine
SRDIVMTQSQKFMSTSVGDRVSVTCKASQNVGTNVAWYQQTPGQSPKALIYSASYRYSGVPDRFTGSGSGTDFTLTINNVQSEDLAEYFCQQYNSYPRTFGGGTTLEIKRADAAPTVSIFPPSSEQLTSG

SEQ ID NO: 6 - protein, 130 aa, Murine
SRDIVLTQSQKFMSTSVGDRVSVTCKASQNVGTNVAWYQQKPGQSPKALIYSASYRYSGVPDRFTGSGSGTDFTLTISNVQSEDLAEYFCQQYNSYPRTFGGGTKLEIKRADAAPTVSIFPPSSEQLTSG

SEQ ID NO: 7 - protein, 130 aa, Murine
SRDVVMTQTQKFMSTSVGDRVSVTCKASQNVGTNVAWYQQKPGQSPKALIYSASYRYSGVPDRFTGSGSGTDFTLTINNVQSEDLAEYFCQQYNSYPRTFGGGTTLEIKRADAAPTVSIFPPSSEQLTSG

SEQ ID NO: 8 - protein, 130 aa, Murine
SRDIVMTQSQKFMSTSVGDRVSVTCKASQNVGTNVAWYQQKPGQSPDALIYSASYRYSGVPDRFTGSGSGTDFTLTITNVQSEDLADYFCQQYNSYPLTFGSGTKLDLKRADAAPTVSIFPPSSEQLTSG

SEQ ID NO: 9 - protein, 129 aa, Murine
SREIVLTQSPAIMSASPGEKVTMTCRASSSVSYMYWYQQKPGSSPRLLIYDTSNLASGVPARFSGSGSGTSYSLTISRMEAEDAATYYCQQWSGYPLTFGAGTKLELKRADAAPTVSIFPPSSEQLTSG

SEQ ID NO: 10 - protein, 134 aa, Murine
SRDIVMTQSPASLAVSLGQRATISCKASQSVDYDGDNYMNWYQQKPGQPPKLLIYAASNLESGIPARFSGSGSGTDFTLNIHPVEEEDAATYYCQQSNGDPWTFGGGTKLEIKRADAAPTVSIFPPSSEQLTSG

SEQ ID NO: 11 - protein, 134 aa, Murine
SRDIVLTQSPASLTVSLGQRATISCRASESVDNYGISFMNWFQQKPGQPPKLLIYAASNQGSGVPARFSGSGSGTDFSLNIHPMEEDDTAMYFCQQTKEVPYTFGGGTKLEIKRADAAPTVSIFPPSSEQLTSG

SEQ ID NO: 12 - protein, 136 aa, Murine
SRDIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPFTFGSGTKLEIKRADAAPTVSIFPPSSEQLTSG

SEQ ID NO: 13 - protein, 130 aa, Murine
  VARIANT 2, 5: Xaa = Any Amino Acid
SXDIXMTQSPASLSVSVGETVTITCRASENIYSNLAWYQQKQGKSPQLLVYAATNLADGVPSRFSGSGSGTQYSLKINSLQSEDFGSYYCQHFWGTPWTFGGGTKLEIKRADAAPTVSIFPPSSEQLTSG

SEQ ID NO: 14 - protein, 130 aa, Murine
SRDIVMTQSQKFMSTSVGDRVTVTCKASQNVGTNVVWYQQKPGHSPKALIYSASYRFGGVPDRFTGSGSGTDFTLTISNVQSEDLAEYFCQQYNIYPYTFGGGTKLEIKRADAAPTVSIFPPSSEQLTSG

SEQ ID NO: 15 - protein, 134 aa, Murine
SRDIVLTQSPASLSVSLGQRATISCKASQSVDNDGISYMNWYQQKPGQPPKLLIYAASNLGSGVPARFSGSGSGTDFSLNIHPVEEEDAATYFCQQYNGYPYTFGGGTKLEIKRADAAPTVSIFPPSSEQLTSG

SEQ ID NO: 16 - protein, 131 aa, Murine
SRDNVLTQSPAIMSASPGEKVTMTCRASSSVGSSYLHWYQQKSGASPKLWIYSTSKLASGVPARFSGSGSGTSYSLTISSVEAEDAATYYCQQYSGYPLTFGGGTKLEIKRADAAPTVSIFPPSSEQLTSG

SEQ ID NO: 17 - protein, 201 aa, Murine
LEVQLQQSGAELMKPGASVKISCKATGYTFSSYWIEWVKQRPGHGLEWIGEILPGIGTTHYNERFKGKATFTADTSSKTVYMQLSSLTSEDSAVYYCVRKNYDWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 18 - protein, 199 aa, Murine
LEVQLQQSGSVLARPGASVKMSCKASGYSFANYWMHWVKQRPGQGLEWIGAIYPGNTDTSYNQKFKGRAKLTAVTSATAYMELNSLTNEDSAVYYCTRLRPPFNFWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCN

SEQ ID NO: 19 - protein, 203 aa, Murine
LEVELQQSGAELVKPGASVKLSCKASGYTFTNYYMHWVKQRPGQGLEWIGEINPSSGGTNFNEKFKSKATLTVDKSSSTAYMQLSSLTSEDSAVYYCTRFDRTENGMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 20 - protein, 203 aa, Murine
LEVQLQQPGAELVKPGASVKMSCKASGYTFTSYYMYWVKQRPGQGLEWIGEINPSHGGTNFNEKFKNKATLTVDKSSSTVYMQLSSLTSEDSAVYYCTRGGNYPYFAMDYWGQGTSVTVSSAKTPPSVYPLAPGSAAQTNSMITLGCLVKGYFPEPVTVTWNSGSLSSGVHTFPAVLQSDLYTLSSSVTVPSSTWPSETVTCN

SEQ ID NO: 21 - protein, 201 aa, Murine
LEVQLQQSGAELVKPGASVKLSCTASGFNIKDTYIHWMNQRPEQGLEWIGRIDPADGNTKYDPKFQDKATITADTSSNTAYLHLSSLTSEDTAVYYCTTAFYYSMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 22 - protein, 201 aa, Murine
LEVQLQQSGAVLLKPGASVKLSCTASGFNIKDTYIHWMKQRPEQGLEWIGRIDPADGNTKYDPKFQGKATITADTSSNTAYLQLSSLTSEDTAVYYCTTAFYYSMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 23 - protein, 201 aa, Murine
LEVQLQQSGAVLLKPGASVKLSCTASGFNIKDTYIHWMKQRPEQGLEWIGRIDPADGNTKYDPKFQGKATITAATSSNTAYLQLSSLTSEDTAVYYCTTAFYYSMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 24 - protein, 200 aa, Murine
LEVQLQQSGPELVKPGASVKISCKTSGYTFTEYTMHWVKQSHGKSLEWIGGINPNNGGTNYNQKFKGKATLTVDKSSSTAYMELRSLTSEDSAVYYCARWTGDFDVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 25 - protein, 200 aa, Murine
LEVQLQQSGSVLARPGSSVKMSCKASGYSFTSYWMHWVKQRPGQGLEWIGSIYPGNSDTSYNQKFKGRAKLTAVTSASTAYMELNSLTSEDSAVYYCTRLRPPFNFWGQGTTLTVSSAKTTAPSVYPLAPVCGDTTGSSMTLGCLVKGYFPEPVTLTWNSGSLSSGVHTFPAVLQSDLYTLSSSVTVTSSTWPSQSITCN

SEQ ID NO: 26 - protein, 203 aa, Murine
LEVQLQQSGAELVKPGASVKLSCKASGYTFTNYYMHWVKQRPGQGLEWIGEINPSSGGTNFNEKFKSKATLTVDKSSSTAYMQLSSLTSEDSAVYYCTRFDRTENGLDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 27 - protein, 204 aa, Murine
LEVQLQQSGSELMKPGASVKISCKATGFTFSSSWIEWVKQRPGHGLEWIGEISPGSGSTNFNENFKGKATLTADTSSNTAYMQLSSLTSEDSAVYYCARFYGNNLYYFDYWGQGTTLTVSSAKTTPPSVYPLAPGSAAQTNSIVTLGCLVKGYFPEPVTVTWNSGSLSSGVHTFPAVLQSDLYTLSSSVTVPSSTWPSETVTCN

SEQ ID NO: 28 - protein, 205 aa, Murine
LEVQLQQSGPELVKTGASVKISCKASGFSITGYYMHWVKQSHGKGLEWIGYISSYSLATDYNQNFKGKATFTVDTSSTTAYMQFNSLTPEDSAVYYCARGDYASPYWFFDVWGAGTAVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 29 - protein, 193 aa, Murine
LKPSQSLSLTCSVTGYSITGGYYWNWIRQFPGNKLEWMGYIRYDGSNNYNPSLKNRISITRDTSKNQFFLKLNSVTTEDTATYYCARGGYDGLYYAMDYWGQGTSVTVSSAKTTAPSVYPLAPVCGDTTGSSMTLGCLVKGYFPEPVTLTWNSGSLSSGVHTFPAVLQSDLYTLSSSVTVTSSTWPSQSITCN

SEQ ID NO: 30 - protein, 157 aa, Murine
PGAELVKPGASVKLSCTASGFNIKDTFLHWVKQRPEQGLEWIGRIDPAKDDTKYDPKLQGKATMTADTSSNTAYLQLSSLTSEDTAVYYCARSTLGRAFAYWGQGTLVTVSAAKTTAPSVYPLAPVYGDTTGSSVTLGCLVKGYFPEPVTLTWNSGS

SEQ ID NO: 31 - protein, 199 aa, Murine
LEVQLQQSGAELARPGASVKMSCKASGNTFNTIHWIKQRPGQGLEWIGYINPSNGLTKNNQKFKDKATLTADKSSSTAYMQLSSLTSEDSAVYSCALGYFYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCN

SEQ ID NO: 32 - protein, 199 aa, Murine; identical to SEQ ID NO: 31

SEQ ID NO: 33 - protein, 11 aa, Murine
RASQDISNYLN

SEQ ID NO: 34 - protein, 10 aa, Murine
SASSSVSYMY

SEQ ID NO: 35 - protein, 15 aa, Murine
KASQSVDYDGDNYMN

SEQ ID NO: 36 - protein, 11 aa, Murine
KASQNVGTNVA

SEQ ID NO: 37 - protein, 10 aa, Murine
RASSSVSYMY

SEQ ID NO: 38 - protein, 15 aa, Murine
RASESVDNYGISFMN

SEQ ID NO: 39 - protein, 17 aa, Murine
KSSQSLLYSSNQKNYLA

SEQ ID NO: 40 - protein, 11 aa, Murine
RASENIYSNLA

SEQ ID NO: 41 - protein, 11 aa, Murine
KASQNVGTNVV

SEQ ID NO: 42 - protein, 15 aa, Murine
KASQSVDNDGISYMN

SEQ ID NO: 43 - protein, 12 aa, Murine
RASSSVGSSYLH

SEQ ID NO: 44 - protein, 7 aa, Murine
YTSRLHS

SEQ ID NO: 45 - protein, 7 aa, Murine
DTSNLAS

SEQ ID NO: 46 - protein, 7 aa, Murine
AASNLES

SEQ ID NO: 47 - protein, 7 aa, Murine
SASYRYS

SEQ ID NO: 48 - protein, 7 aa, Murine
AASNQGS

SEQ ID NO: 49 - protein, 7 aa, Murine
WASTRES

SEQ ID NO: 50 - protein, 7 aa, Murine
AATNLAD

SEQ ID NO: 51 - protein, 7 aa, Murine
SASYRFG

SEQ ID NO: 52 - protein, 7 aa, Murine
AASNLGS

SEQ ID NO: 53 - protein, 7 aa, Murine
STSKLAS

SEQ ID NO: 54 - protein, 9 aa, Murine
QQGNTLPYT

SEQ ID NO: 55 - protein, 9 aa, Murine
QQWSSYPLT

SEQ ID NO: 56 - protein, 9 aa, Murine
QQSDEDPYT

SEQ ID NO: 57 - protein, 9 aa, Murine
QQGNTLPWT

SEQ ID NO: 58 - protein, 9 aa, Murine
QQYNSYPRT

SEQ ID NO: 59 - protein, 9 aa, Murine
QQYNSYPLT

SEQ ID NO: 60 - protein, 9 aa, Murine
QQWSGYPLT

SEQ ID NO: 61 - protein, 9 aa, Murine
QQSNGDPWT

SEQ ID NO: 62 - protein, 9 aa, Murine
QQTKEVPYT

SEQ ID NO: 63 - protein, 9 aa, Murine
QQYYSYPFT

SEQ ID NO: 64 - protein, 9 aa, Murine
QHFWGTPWT

SEQ ID NO: 65 - protein, 9 aa, Murine
QQYNIYPYT

SEQ ID NO: 66 - protein, 9 aa, Murine
QQYNGYPYT

SEQ ID NO: 67 - protein, 9 aa, Murine
QQYSGYPLT

SEQ ID NO: 68 - protein, 10 aa, Murine
GYTFSSYWIE

SEQ ID NO: 69 - protein, 10 aa, Murine
GYSFANYWMH

SEQ ID NO: 70 - protein, 10 aa, Murine
GYTFTNYYMH

SEQ ID NO: 71 - protein, 10 aa, Murine
GYTFTSYYMY

SEQ ID NO: 72 - protein, 10 aa, Murine
GFNIKDTYIH

SEQ ID NO: 73 - protein, 10 aa, Murine
GYTFTEYTMH

SEQ ID NO: 74 - protein, 10 aa, Murine
GYSFTSYWMH

SEQ ID NO: 75 - protein, 10 aa, Murine
GFTFSSSWIE

SEQ ID NO: 76 - protein, 10 aa, Murine
GFSITGYYMH

SEQ ID NO: 77 - protein, 11 aa, Murine
GYSITGGYYWN

SEQ ID NO: 78 - protein, 10 aa, Murine
GFNIKDTFLH

SEQ ID NO: 79 - protein, 8 aa, Murine
GNTFNTIH

SEQ ID NO: 80 - protein, 17 aa, Murine
EILPGIGTTHYNERFKG

SEQ ID NO: 81 - protein, 17 aa, Murine
AIYPGNTDTSYNQKFKG

SEQ ID NO: 82 - protein, 17 aa, Murine
EINPSSGGTNFNEKFKS

SEQ ID NO: 83 - protein, 17 aa, Murine
EINPSHGGTNFNEKFKN

SEQ ID NO: 84 - protein, 17 aa, Murine
RIDPADGNTKYDPKFQD

SEQ ID NO: 85 - protein, 17 aa, Murine
RIDPADGNTKYDPKFQG

SEQ ID NO: 86 - protein, 17 aa, Murine
GINPNNGGTNYNQKFKG

SEQ ID NO: 87 - protein, 17 aa, Murine
SIYPGNSDTSYNQKFKG

SEQ ID NO: 88 - protein, 17 aa, Murine
EISPGSGSTNFNENFKG

SEQ ID NO: 89 - protein, 17 aa, Murine
YISSYSLATDYNQNFKG

SEQ ID NO: 90 - protein, 16 aa, Murine
YIRYDGSNNYNPSLKN

SEQ ID NO: 91 - protein, 17 aa, Murine
RIDPAKDDTKYDPKLQG

SEQ ID NO: 92 - protein, 17 aa, Murine
YINPSNGLTKNNQKFKD

SEQ ID NO: 93 - protein, 8 aa, Murine
KNYDWFAY

SEQ ID NO: 94 - protein, 7 aa, Murine
LRPPFNF

SEQ ID NO: 95 - protein, 10 aa, Murine
FDRTENGMDY

SEQ ID NO: 96 - protein, 11 aa, Murine
GGNYPYFAMDY

SEQ ID NO: 97 - protein, 8 aa, Murine
AFYYSMDY

SEQ ID NO: 98 - protein, 7 aa, Murine
WTGDFDV

SEQ ID NO: 99 - protein, 10 aa, Murine
FDRTENGLDY

SEQ ID NO: 100 - protein, 11 aa, Murine
FYGNNLYYFDY

SEQ ID NO: 101 - protein, 12 aa, Murine
GDYASPYWFFDV

SEQ ID NO: 102 - protein, 12 aa, Murine
GGYDGLYYAMDY

SEQ ID NO: 103 - protein, 9 aa, Murine
STLGRAFAY

SEQ ID NO: 104 - protein, 8 aa, Murine
GYFYAMDY

(19) United States
(12) Patent Application Publication
(10) Pub. No.: US 2008/0287309 A1
(43) Pub. Date: Nov. 20, 2008

(54) Methods for Discovering Antibodies Specific to Cancer Cells and Antibodies Discovered Thereby

(75) Inventors: Katherine S. Bowdish, Del Mar, CA (US); Hong Xin, Bonsall, CA (US); Ferda Yantiri-Wernimont, Oceanside, CA (US); Amara Siva, Oceanside, CA (US)

Correspondence Address: ROPES & GRAY LLP, PATENT DOCKETING 39/41, ONE INTERNATIONAL PLACE, BOSTON, MA, 02110-2624, US

(73) Assignee: Alexion Pharmaceuticals, Inc., Cheshire, CT (US)

(21) Appl. No.: 11/631,911

(22) PCT Filed: Jul. 8, 2005
(86) PCT No.: PCT/US05/24260

§ 371 Date: Jan. 11, 2008

Related U.S. Application Data
(60) Provisional application No. 60/586,811, filed on Jul. 10, 2004.

(51) Int. Cl. C40B 30/04; A01N 1/02; C07K 16/18; C12N 15/11; C12N 15/00; C12N 5/06

(52) U.S. Cl. 506/9; 435/2; 530/387.7; 536/23.53; 435/320.1; 435/325

(57) ABSTRACT

This disclosure relates to methods for selecting antibodies having desirable characteristics from a population of diverse antibodies. More specifically, this disclosure provides methods for identifying antibodies which bind to cancer cells, but which do not bind to human red blood cells, white blood cells or normal tissue cells. Antibodies of the disclosure can be used for therapeutic and/or diagnostic purposes.

### RELATED APPLICATION

This application claims priority to U.S. Provisional Patent Application Ser. No. 60/586,811, filed on Jul. 10, 2004, the disclosure of which is incorporated herein in its entirety.

### TECHNICAL FIELD

This disclosure relates to methods for selecting antibodies having desirable characteristics from a population of diverse antibodies. More specifically, this disclosure provides methods for identifying antibodies which bind to cancer cells, but which do not bind to human red blood cells, white blood cells or normal tissue cells.

### BACKGROUND OF RELATED ART

The promise of monoclonal antibody therapy is beginning to be realized. Efficacy has been seen in clinical trials using antibodies that target tumor cell surface antigens such as B-cell idiotypes, CD20 on malignant B cells, CD33 on leukemic blasts, and HER2/neu on breast cancer. Trastuzumab (Herceptin, anti-HER2/neu, Genentech) leads to objective responses in some metastatic breast cancer patients with overexpression of the HER2/neu oncoprotein. These exciting results provide a basis for further refinement of the existing approaches to develop new antibody-based cancer therapy strategies. Recent clinical results of monoclonal antibodies in combination with or without chemotherapy, including Erbitux (Cetuximab, C225, anti-EGFr, ImClone) in the treatment of metastatic colon cancer and Bevacizumab (Avastin, anti-vEGFr, Genentech) in the treatment of colon, renal cell cancer and other solid tumors, strongly demonstrate that monoclonal antibodies can be beneficial for cancer patients. Currently, there are multiple clinical trials with monoclonal antibodies for the treatment of prostate cancer.

Generation of murine monoclonal antibodies with hybridoma technology, phage display, or other technologies, such as ribosomal display and yeast display, is especially critical for both basic and clinical sciences. Herceptin, Erbitux and Bevacizumab were originally screened from antigen-immunized mice.

Much research has been done to discover antibodies against cancer cells through whole cell immunization followed by screening antibodies, which bind to surface molecules of cancer cells. Although the theory of this approach is very attractive, few therapeutic antibodies were found after years of effort. This approach has proven difficult for several reasons. One reason is that the immune response in mice is not tumor specific even though cancer cells are used as an immunogen because cancer cells share a lot of common surface antigens with normal cells. Thus, the screening for tumor specific antibodies could prove to be very difficult and/or fruitless.

It is a general phenomenon that cancer cells share common antigens with normal cells. In the past, negative and positive selections have been used to screen for tumor specific antibodies. To facilitate screening for tumor specific antibodies, negative selection is a general method used to address the problem of antigens common to both normal and cancer cells, which interferes with positive selections. Numerous publications have used normal tissue cells to subtract undesired antibodies that bind to common antigens on both cancer cells and normal tissues. See, Zijlstra, et al. Biochem Biophys Res Commun. 2003 Apr. 11; 303(3):733-44; Hooper et al., Oncogene. 2003 Mar. 27; 22(12):1783-94; and Foss, Semin Oncol. 2002 June; 29(3 Suppl 7):5-11. However, most of these publications have used only one type of normal tissue cell or a couple of normal cell lines for subtraction.

Previous attempts were also made to solve this problem by an alternative method called subtractive immunization. Intensive research has been done with subtractive immunization in the past 15 years. Subtractive immunization focuses on the immunization step instead of the whole cell panning step. Subtractive immunization utilizes a distinct immune tolerization approach that can enhance the generation of monoclonal antibodies to desired antigens. Subtractive immunization is based on tolerizing the host animal to immunodominant or otherwise undesired antigens that may be structurally or functionally related to the antigens of interest. Tolerization of the host animal can be achieved through one of three methods: High Zone, Neonatal, or Drug-induced tolerization. The tolerized animal is then inoculated with the desired antigens and antibodies generated by the subsequent immune response are screened for the desired reactivity. However, a recent study suggested that neonatal “tolerization” induces immune deviation, not tolerance in the immunological sense. Neonates are not immune-privileged but generate T_{H}2 or T_{H}1 responses, depending on the mode of immunization. The chemical immunosuppression with cyclophosphamide was the most effective subtractive immunization technique. As those skilled in the art will appreciate, normal cell immunization followed by cyclophosphamide treatment will kill all the proliferating immune cells reactive with normal cell antigens. However, this regimen also kills all of the helper T-cells required for B-cell maturation and differentiation. Therefore, when this regimen is followed by cancer cell immunization to elicit antibodies specific to tumor antigens, only low affinity antibodies of IgM isotype are produced.

It would be advantageous to have improved methods for screening antibody libraries to identify antibodies which bind to surface molecules of cancer cells. Improved methods for treating individuals suffering from cancer are also desirable.

### SUMMARY

Antibodies that bind to cancer cells but not to normal cells are identified using a negative selection process. A library of antibodies created by immunization of an animal with cancer cells is contacted with red blood cells and/or white blood cells and, optionally on other normal (i.e., non-cancerous) cells. The blood and/or normal cells, along with the antibodies that bind to those cells are removed, leaving a sub-library of antibodies that can be panned against cancer cells to identify antibodies that bind to the cancer cells, but (due to clearing effect of the negative selection process) show little to no binding to normal cells. These antibodies can be used for therapeutic and/or diagnostic purposes.

Thus, in one embodiment the present methods include the steps of collecting antiserum from subjects immunized with a cancer cell; contacting the antiserum with human blood cells (red and/or white) and optionally normal tissue cells; and recovering the portion of the antiserum that does not bind to the human red blood cells. In another embodiment, antiserum from subjects immunized with a cancer cell is collected; antibodies that bind to human blood cells (red and/or white) and optionally normal tissue cells are removed from the antiserum; and antibodies that bind to the cancer cell are recovered from the antiserum. In yet another embodiment the present methods include the steps of collecting antiserum from subjects immunized with a cancer cell; removing antibodies that bind to human red blood cells and antibodies that bind to at least one other type of non-cancerous cell from the antiserum and then recovering from the antiserum antibodies that bind to the cancer cell.

In a particularly useful embodiment, the methods include the steps of generating a phage displayed antibody library using cells collected from subjects immunized with cancer cells; removing members of the library that bind to human red blood cells to generate a sub-library; and recovering from the sub-library members that display antibodies that bind to the cancer cell.

In another embodiment, the present disclosure relates to an antibody that binds to a prostate cancer cell comprising either: a light Chain CDR1 selected from the group consisting of RASQDISNYLN (SEQ ID NO: 33), SASSSVSYMY (SEQ ID NO: 34), KASQSVDYDGDNYMN (SEQ ID NO: 35), KASQNVGTNVA (SEQ ID NO: 36), RASSSVSYMY (SEQ ID NO: 37), RASESVDNYGISFMN (SEQ ID NO: 38), KSSQSLLYSSNQKNYLA (SEQ ID NO: 39), RASENIYSNLA (SEQ ID NO: 40), KASQNVGTNVV (SEQ ID NO: 41), KASQSVDNDGISYMN (SEQ ID NO: 42), and RASSSVGSSYLH (SEQ ID NO: 43); a light chain CDR2 selected from the group consisting of YTSRLHS (SEQ ID NO: 44), DTSNLAS (SEQ ID NO: 45), AASNLES (SEQ ID NO: 46), SASYRYS (SEQ ID NO: 47), AASNQGS (SEQ ID NO: 48), WASTRES (SEQ ID NO: 49), AATNLAD (SEQ ID NO: 50), SASYRFG (SEQ ID NO: 51), AASNLGS (SEQ ID NO: 52), and STSKLAS (SEQ ID NO: 53); a light chain CDR3 selected from the group consisting of QQGNTLPYT (SEQ ID NO: 54), QQWSSYPLT (SEQ ID NO: 55), QQSDEDPYT (SEQ ID NO: 56), QQGNTLPWT (SEQ ID NO: 57), QQYNSYPRT (SEQ ID NO: 58), QQYNSYPLT (SEQ ID NO: 59), QQWSGYPLT (SEQ ID NO: 60), QQSNGDPVWT (SEQ ID NO: 61), QQTKEVPYT (SEQ ID NO: 62), QQYYSYPFT (SEQ ID NO: 63), QHFWGTPWT (SEQ ID NO: 64), QQYNIYPYT (SEQ ID NO: 65), QQYNGYPYT (SEQ ID NO: 66), and QQYSGYPLT (SEQ ID NO: 67); a heavy chain CDR1 selected from the group consisting of GYTFSSYWIE (SEQ ID NO: 68), GYSFANYWMH (SEQ ID NO: 69), GYTFTNYYMH (SEQ ID NO: 70), GYTFTSYYMY (SEQ ID NO: 71), GFNIKDTYIH (SEQ ID NO: 72), GYTFTEYTMH (SEQ ID NO: 73), GYSFTSYWMH (SEQ ID NO: 74), GFTFSSSWIE (SEQ ID NO: 75), GFSITGYYMH (SEQ ID NO: 76), GYSITGGYYWN (SEQ ID NO: 77), GFNIKDTFLH (SEQ ID NO: 78), and GNTFNTIH (SEQ ID NO: 79); a heavy chain CDR2 selected from the group consisting of EILPGIGTTHYNERFKG (SEQ ID NO: 80), AIYPGNTDTSYNQKFKG (SEQ ID NO: 81), EINPSSGGTNFNEKFKS (SEQ ID NO: 82), EINPSHGGTNFNEKFKN (SEQ ID NO: 83), RIDPADGNTKYDPKFQD (SEQ ID NO: 84), RIDPADGNTKYDPKFQG (SEQ ID NO: 85), GINPNNGGTNYNQKFKG (SEQ ID NO: 86), SIYPGNSDTSYNQKFKG (SEQ ID NO: 87), EISPGSGSTNFNENFKG (SEQ ID NO: 88), YISSYSLATDYNQNFKG (SEQ ID NO: 89), YIRYDGSNNYNPSLKN (SEQ ID NO: 90), RIDPAKDDTKYDPKLQG (SEQ ID NO: 91), and YINPSNGLTKNNQKFKD (SEQ ID NO: 92); or a heavy chain CDR3 selected from the group consisting of KNYDWFAY (SEQ ID NO: 93), LRPPFNF (SEQ ID NO: 94), FDRTENGMDY (SEQ ID NO: 95), GGNYPYFAMDY (SEQ ID NO: 96), AFYYSMDY (SEQ ID NO: 97), WTGDFDV (SEQ ID NO: 98), FDRTENGLDY (SEQ ID NO: 99), FYGNNLYYFDY (SEQ ID NO: 100), GDYASPYWFFDV (SEQ ID NO: 101), GGYDGLYYAMDY (SEQ ID NO: 102), STLGRAFAY (SEQ ID NO: 103), and GYFYAMDY (SEQ ID NO: 104).

In other embodiments, the present disclosure relates to an antibody that binds to a prostate cancer cell comprising an amino acid sequence selected from the group consisting of SEQ ID NOS: 1-16.

Isolated nucleic acid encoding any of foregoing antibodies, expression vectors containing such isolated nucleic acid and host cells transfected with such expression vectors are also contemplated.

In yet another embodiment, the present disclosure relates to a method that includes the steps of contacting cancer cells with a hapten (such as, for example dinitrophenyl); generating a phage displayed antibody library using cells collected from subjects immunized with the cancer cells; removing members of the library that bind to human red blood cells to generate a sub-library; and recovering from the sub-library members that display antibodies that bind to the cancer cell.

### BRIEF DESCRIPTION OF THE DRAWINGS

For a fuller understanding of the subject matter described herein, reference should be made to the following detailed description, taken in connection with the accompanying diagrammatic drawings, in which:

FIG. 1 shows the results of FACS analyses of anticancer sera subtracted with human red blood cells.

FIG. 2 schematically shows the steps involved in the panning of an antibody library, subtracting out the antibodies that bind to normal cells and screening for antibodies that bind to cancerous cells.

FIG. 3 shows the antigen signature for PC3 antibodies with linear epitopes.

FIG. 4A shows the amino acid sequences of antibody light chains (SEQ ID NOS: 1 THROUGH 16) identified using the process of FIG. 2.

FIG. 4B shows the amino acid sequences of antibody heavy chains (SEQ ID NOS: 17 THROUGH 32) identified using the process of FIG. 2.

### DETAILED DESCRIPTION OF PREFERRED EMBODIMENTS

Stringent negative selection is used in accordance with this disclosure to screen for tumor specific antibodies. The stringent negative selection strategy in accordance with this disclosure includes multi-step subtractions with human blood cells and, optionally normal tissue cells during the whole cell panning. The present methods significantly decrease the number of selected antibodies that bind to normal human cells, especially blood cells. These methods show improved antibody diversity by a whole cell panning approach, and provide a way to select tumor specific antibodies for cancer diagnostics and therapeutics. For therapeutic purposes, antibodies identified in accordance with the methods described herein will likely have reduced side effects on normal blood cells. This feature should improve the safety profile of the antibody for cancer therapy.

As used herein, the term “antibodies” refers to complete antibodies or antibody fragments capable of binding to a selected target. Included are Fv, scFv, Fab′ and F(ab′)2, monoclonal and polyclonal antibodies, engineered antibodies (including chimeric, CDR-grafted and humanized, fully human antibodies, and artificially selected antibodies), and synthetic or semi-synthetic antibodies produced using phage display or alternative techniques. Small fragments, such as Fv and scFv, possess advantageous properties for diagnostic and therapeutic applications on account of their small size and consequent superior tissue distribution.

The present antibodies are identified by screening an antibody library. Techniques for producing an antibody library are within the purview of one skilled in the art. See, Rader and Barbas, Phage Display, A Laboratory Manual, Cold Spring Harbor Laboratory Press, Cold Spring Harbor, N.Y. (2000), U.S. Pat. No. 6,291,161 to Lerner et al. and copending, published U.S. Patent Applications US20040072164A1 and US20040101886A1, the disclosures of which are incorporated herein in their entirety by this reference. Antibodies can be raised in a subject, for example, by one or more injections of an immunizing agent and, if desired, an adjuvant. The immunizing agent may include any type of cancer cell or fragments thereof. Typically, the immunizing agent and/or adjuvant will be injected in the subject by multiple subcutaneous or intraperitoneal injections. Suitable adjuvants include, but are not limited to adjuvants that have been used in connection with cancer cell vaccines, such as, for example, unmethylated CpG motifs and Bacillus Calmette-Guerin (BCG). The immunization protocol may be selected by one skilled in the art without undue experimentation.

Any type of cancer cell can be used for immunizing a subject in accordance with the present methods. Suitable types of cancer cells include, but are not limited to hematopoetic malignancies, melanoma, breast, ovarian, prostate, colon, head and neck, lung, renal, stomach, pancreatic, liver, bladder and brain. Cancer cells can be obtained from a variety of sources. For example, primary samples of cancer cells can be obtained directly from patients either through surgical techniques or biopsies. Cancer cells are also available from National Development and Research Institutes, Inc. (“NDRI”), New York, N.Y. Various types of cancer cells have also been deposited with and are available from American Type Culture Collection, Manassas, Va. (“ATCC”) or other depositories, such as the National Cancer Institute. Where fragments of cancer cells (such as cell membranes or mitochondria) are to be used as the immunizing agent, techniques within the purview of those skilled in the art may be employed to disrupt the cancer cells and isolate suitable components for use in immunization.

In certain embodiments, enhancement of antibody response to epitopes on the cancer cells is achieved by modification with a hapten, such as dinitrophenyl (DNP). DNP is a highly immunogenic hapten, which makes the cancer cells more easily recognized by the immune system. DNP is an aromatic compound (benzene ring with disubstituted nitro groups) that has the configuration of a hapten. A hapten is an antigenic determinant that is capable of binding to an antibody but incapable of eliciting an antibody response on its own but does when linked to a carrier protein. DNP modified autologous cancer cell vaccines have been shown to elicit a robust immune response, which is characterized by delayed type hypersensitivity, release of proinflammatory cytokines such as IFN-γ and expansion of both CD4 and CD8 T cell subsets. DNP modification of low-density antigens preferentially attract B-cells to the site of immunogen and allow recognition and expansion of B-cells in response to DNP modified antigen. The process of B-cell trafficking to the immunogen and their subsequent expansion can be further aided by release of proinflammatory cytokines. DNP modification can be accomplished using techniques within the purview of those skilled in the art, such as those described in Berd, et al., J Clin Oncol 22:403 (2004); and Sojka, et al., Cancer Immunol Immunother 1:200 (2002).

Once an immune response is elicited in the subject, antibodies may be collected for the selection process. Cells from tissue that produce or contain antibodies are collected from the subject about three to five days after the last immunization. Suitable tissues include blood, spleen, lymph nodes and bone marrow.

Once the cells are collected, RNA is isolated therefrom using techniques known to those skilled in the art and a combinatorial antibody library is prepared. In general, techniques for preparing a combinatorial antibody library involve amplifying target sequences encoding antibodies or portions thereof, such as, for example the light and/or heavy chains using the isolated RNA of an antibody. Thus, for example, starting with a sample of antibody mRNA that is naturally diverse, first strand cDNA can be produced to provide a template. Conventional PCR or other amplification techniques can then be employed to generate the library. In certain embodiments, phage libraries expressing antibody Fab fragments (kappa or lambda light chains complexed to the IgG heavy chain fragment (Fd) are constructed in plasmid vectors using the methods described in U.S. application Ser. No. 10/251,085, the disclosure of which is incorporated herein in its entirety by this reference.

The phage display library can then be assayed for the presence of antibodies directed against the cancer cells. Preferably, the binding specificity of antibodies is determined by an in vitro binding assay such as enzyme-linked immunoabsorbent assay (ELISA) and/or fluorescence-activated cell sorting (FACS). Such techniques and assays are known in the art. The binding affinity of an antibody can, for example, be determined by the Scatchard analysis of Munson and Pollard, Anal. Biochem., 107:220 (1980).

In accordance with the methods described herein, after conducting positive selection on cancer cells, human blood cells (either red, white or both), and optionally normal (i.e., non-cancerous) tissue cells are used as absorbers in conducting stringent subtractions prior to screening of the library. Suitable human normal tissue cells for use in the subtraction process include endothelia cells, epithelial cells, smooth muscle cells, and other cells isolated from such tissues as liver, lung, heart, kidney, intestine, stomach, bladder, spleen, pancreas, bone marrow, brain, thymus, prostate, ovary, testis, skin, and the like. Suitable tissue can be obtained, for example, from normal donors, late stage of fetus, or from cell lines established from these tissues.

The subtractions can be performed by contacting the library of antibodies with the normal cells and then removing the normal cells along with any antibodies bound thereto. Removal of the cells can be achieved using any technique within the purview of those skilled in the art, such as centrifuging. The supernatant containing the unbound antibodies is retained as it is the portion that contains a sub-library of antibodies that bind to cancer cells but not to normal cells. To help ensure that all antibodies that bind to normal cells are removed, multiple rounds of subtraction are performed. The multiple rounds can be conducted using the same or different types of cells. In particularly useful embodiments, at least three rounds of subtraction using red blood cells are performed. In one embodiment, subtraction is done with both red blood cells (3 rounds with different blood types (e.g., A type, B type, etc.)) and white blood cells (one round). In other embodiments, multiple subtractions are conducted using at least two types of non-cancerous cells; namely, at least one type of blood cell and at least one other type of normal tissue cells. Advantageously, the normal tissue can be derived from the same type of tissue as the cancer cells used for immunization. For example, if the subject was immunized with pancreatic cancer cells, then normal (i.e., non-cancerous) pancreatic tissue cells are used to perform the subtractions.

In conducting the negative selection, the ratio of antibody phage versus red blood cells or other absorber cells can be selected by one skilled in the art without undue experimentation. In certain embodiments, 700-1000 phage per red blood cell can be used.

To provide adequate numbers of library members, the sub-library can be amplified between rounds of subtraction and/or prior to the screening for antibodies that bind to cancer cells. Techniques for amplification are within the purview of those skilled in the art.

After the negative selection process, antibodies derived from recombinant libraries may be selected using cancer cells, or polypeptides derived therefrom, to isolate the antibodies on the basis of target specificity. As noted above, suitable techniques for selecting antibodies that bind to cancer cells are within the purview of those skilled in the art.

Hybridoma methods can also be used to identify antibodies having the desired characteristics. Such techniques are within the purview off those skilled in the art. In a hybridoma method, a mouse, rabbit, rat, hamster, or other appropriate host animal, is typically immunized with cancer cells (masked as described in copending International Application No. ______ entitled “Antibodies Against Cancer Produced Using Masked Cancer Cells As Immunogen” filed under Express Mail Label No. EL983568278US on Jul. 8, 2005, the disclosure of which is incorporated herein in its entirety) to elicit lymphocytes that produce or are capable of producing antibodies that will specifically bind to the cancer cells. Alternatively, the lymphocytes may be immunized in vitro. The lymphocytes are then fused with an immortalized cell line using a suitable fusing agent, such as polyethylene glycol, to form a hybridoma cell (See, Goding, Monoclonal Antibodies: Principles and Practice, Academic Press, (1986) pp. 59-103; Kozbor, J. Immunol., 133:3001 (1984); and Brodeur et al., Monoclonal Antibody Production Techniques and Applications, Marcel Dekker, Inc., New York, (1987) pp. 51-63 the disclosures of which are incorporated herein by this reference). The hybridoma cells are cultured in a suitable culture medium that preferably contains one or more substances that inhibit the growth or survival of the unfused, immortalized cells. The culture medium in which the hybridoma cells are cultured can then be assayed for the presence of monoclonal antibodies directed against the cancer cells using techniques within the purview of those skilled in the art (e.g., FACS analysis) and may be subjected to negative selection in accordance with the methods of the present disclosure. After the desired hybridoma cells are identified, the clones may be subcloned by limiting dilution procedures and grown by standard methods. Alternatively, the hybridoma cells may be grown in vivo as ascites in a mammal. The monoclonal antibodies secreted by the subclones are isolated or purified from the culture medium or ascites fluid by conventional immunoglobulin purification procedures.

The monoclonal antibodies that bind to cancer cells but show little or no binding to normal cells can be made by recombinant DNA methods that are within the purview of those skilled in the art. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells or phage (depending on the particular selection method employed to identify the antibody) may serve as a preferred source of such DNA. Once isolated, the DNA may be placed into expression vectors, which are then transfected into host cells such as simian COS cells, Chinese hamster ovary (CHO) cells, or NSO or other myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences or by covalently joining to the immunoglobulin coding sequence all or part of the coding sequence for a non-immunoglobulin polypeptide.

In a further embodiment, there is provided a method for identifying proteins uniquely expressed in cancer cells employing antibodies in accordance with the present disclosure, by methods well known to those, skilled with art. In one method, Fab or scFv antigens are identified by immunoprecipitation and mass spectrometry. Specifically, in one such method to identify the antigens for these antibodies, scFvs are used to immunoprecipitate the antigens from lysates prepared from the microsomal fraction of cell-surface biotinylated cancer cells. Specifically, cancer cells are labeled with a solution of 0.5 mg/ml sulfo-NHS-LC-biotin in PBS, pH8.0 for 30 seconds. After washing with PBS to remove unreacted biotin, the cells are disrupted by nitrogen cavitation and the microsomal fraction is isolated by differential centrifugation. The microsomal fraction is resuspended in NP40 Lysis Buffer and extensively precleared with normal mouse serum and protein A sepharose. Antigens are immunoprecipitated with HA-tagged scFv antibodies coupled to Rat Anti-HA agarose beads. Following immunoprecipitation, antigens are separated by SDS-PAGE and detected by Western blot using streptavidin-alkaline phosphatase (AP) or by Coomassie G-250 staining. An antibody which does not bind to the cancer cells is used as a negative control. Antigen bands are excised from the Coomassie-stained gel and identified by mass spectrometry (MS). The immunoprecipitated antigens can also be identified by matrix assisted laser desorption ionization mass spectrometry (MALDI-MS) or microcapillary reverse-phase HPLC nano-electrospray tandem mass spectrometry (μLC/MS/MS). The antigens identified can then be used as an immunogen to elicit additional antibodies thereto using techniques within the purview of those skilled in the art.

The present antibodies that bind to cancer cells but show little or no binding to normal cells in accordance with this disclosure may further include humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also include residues which are found neither in the recipient antibody nor in the imported CDR or framework sequences. In general, the humanized antibody will include substantially all of at least one, and typically two, variable domains, in which all or substantially all of the CDR regions correspond to those of one or more non-human immunoglobulins and all or substantially all of the FR regions are those of a human immunoglobulin consensus sequence. The humanized antibody optimally also will include at least a portion of an immunoglobulin constant region (Fc), typically that of a human immunoglobulin (Jones et al., Nature, 321:522-525 (1986); Riechmann et al., Nature, 332:323-329 (1988); and Presta, Curr. Op. Struct. Biol., 2:593-596 (1992)).

Methods for humanizing non-human antibodies are well known in the art. Generally, a humanized antibody has one or more amino acid residues introduced into it from a source which is non-human. These non-human amino acid residues are often referred to as “donor” residues, which are typically taken from a “donor” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which all or some CDR residues and possibly some FR residues are substituted by residues from analogous sites in rodent antibodies.

The present antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking. In vitro methods are also suitable for preparing monovalent antibodies. Digestion of antibodies to produce fragments thereof, particularly, Fab fragments, can be accomplished using routine techniques known in the art.

In other embodiments, bispecific antibodies are contemplated. Bispecific antibodies are monoclonal, preferably human or humanized, antibodies that have binding specificities for at least two different antigens. In the present case, one of the binding specificities is for a cancer cell, the other one is for any other antigen, and preferably for a cell-surface protein or receptor or receptor subunit.

Methods for making bispecific antibodies are within the purview of those skilled in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Antibody variable domains with the desired binding specificities (antibody-antigen combining sites) can be fused to immunoglobulin constant domain sequences. The fusion preferably is with an immunoglobulin heavy-chain constant domain, including at least part of the hinge, CH2, and CH3 regions. DNAs encoding the immunoglobulin heavy-chain fusions and, if desired, the immunoglobulin light chain, are inserted into separate expression vectors, and are co-transfected into a suitable host organism. For further details of illustrative currently known methods for generating bispecific antibodies see, for example, Suresh et al., Methods in Enzymology, 121:210 (1986); WO 96/27011; Brennan et al., Science 229:81 (1985); Shalaby et al., J. Exy. Med. 175:217-225 (1992); Kostelny et al., J. Immunol. 148(5):1547-1553 (1992); Hollinger et al., Proc. Natl. Acad. Sci. USA 90:6444-6448 (1993); and Gruber et al., J. Immunol. 152:5368 (1994); and Tutt et al., J. Immunol. 147:60 (1991).

The present antibodies can be administered as a therapeutic to cancer patients. Because the antibodies exhibit little to no binding to human blood cells or normal tissue cells, reduced side effects can be observed compared to other antibody therapies.

The present antibodies also may be utilized to detect cancerous cells in vivo. This is achieved by labeling the antibody, administering the labeled antibody to a subject, and then imaging the subject. Examples of labels useful for diagnostic imaging in accordance with the present disclosure are radiolabels such as ¹³¹I, ¹¹¹In, ¹²³I, ⁹⁹mTc, ³²P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes, such as a transrectal probe, can also be employed. These isotopes and transrectal detector probes, when used in combination, are especially useful in detecting prostatic fossa recurrences and pelvic nodal disease. The antibody can be labeled with such reagents using techniques known in the art. For example, see Wensel and Meares, Radioimmunoimaging and Radioimmunotherapy, Elsevier, N.Y. (1983), which is hereby incorporated by reference, for techniques relating to the radiolabeling of antibodies. See also, D. Colcher et al., “Use of Monoclonal Antibodies as Radiopharmaceuticals for the Localization of Human Carcinoma Xenografts in Athymic Mice”, Meth. Enzymol. 121: 802-816 (1986), which is hereby incorporated by reference.

A radiolabeled antibody in accordance with this disclosure can be used for in vitro diagnostic tests. The specific activity of a antibody, binding portion thereof, probe, or ligand, depends upon the half-life, the isotopic purity of the radioactive label, and how the label is incorporated into the biological agent. In immunoassay tests, the higher the specific activity, in general, the better the sensitivity. Procedures for labeling antibodies with the radioactive isotopes are generally known in the art.

The radiolabeled antibodies can be administered to a patient where it is localized to the tumor bearing the antigen with which the antibody reacts, and is detected or “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp. 65-85 (Academic Press 1985), which is hereby incorporated by reference. Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ¹³N).

Fluorophore and chromophore labeled biological agents can be prepared from standard moieties known in the art. Since antibodies and other proteins absorb light having wavelengths up to about 310 nm, the fluorescent moieties should be selected to have substantial absorption at wavelengths above 310 nm and preferably above 400 nm. A variety of suitable fluorescers and chromophores are described by Stryer, Science, 162:526 (1968) and Brand, L. et al., Annual Review of Biochemistry, 41:843-868 (1972), which are hereby incorporated by reference. The antibodies can be labeled with fluorescent chromophore groups by conventional procedures such as those disclosed in U.S. Pat. Nos. 3,940,475, 4,289,747, and 4,376,110, which are hereby incorporated by reference.

The present antibodies can also be utilized to kill or ablate cancerous cells in vivo. This involves administering the antibodies bonded to a cytotoxic drug to a subject requiring such treatment. Since the antibodies recognize cancer cells, any such cells to which the antibodies bind are destroyed. Due to the use of the stringent subtraction technique, the amount of normal cells destroyed is minimal.

The antibodies of the present disclosure may be used to deliver a variety of cytotoxic drugs including therapeutic drugs, a compound emitting radiation, molecules of plants, fungal, or bacterial origin, biological proteins, and mixtures thereof. The cytotoxic drugs can be intracellularly acting cytotoxic drugs, such as short-range radiation emitters, including, for example, short-range, high-energy α-emitters. Enzymatically active toxins and fragments thereof are exemplified by diphtheria toxin A fragment, nonbinding active fragments of diphtheria toxin, exotoxin A (from Pseudomonas aeruginosa), ricin A chain, abrin A chain, modeccin A chain, α-sacrin, certain Aleurites fordii proteins, certain Dianthin proteins, Phytolacca americana proteins (PAP, PAPII and PAP-S), Morodica charantia inhibitor, curcin, crotin, Saponaria officinalis inhibitor, gelonin, mitogillin, restrictocin, phenomycin, and enomycin, for example. Procedures for preparing enzymatically active polypeptides of the immunotoxins are described in WO84/03508 and WO85/03508, which are hereby incorporated by reference. Certain cytotoxic moieties are derived from adriamycin, chlorambucil, daunomycin, methotrexate, neocarzinostatin, and platinum, for example.

Procedures for conjugating the antibodies with the cytotoxic agents have been previously described.

Alternatively, the antibody can be coupled to high energy radiation emitters, for example, a radioisotope, such as ¹³¹I, a γ-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (eds.), pp 303-316 (Academic Press 1985), which is hereby incorporated by reference. Other suitable radioisotopes include α-emitters, such as ²¹²Bi, ²¹³Bi, and ²¹¹At, and β-emitters, such as ¹⁸⁶Re and ⁹⁰Y. Radiotherapy is expected to be particularly effective in connection with prostate cancer, because prostate cancer is a relatively radiosensitive tumor.

Where the antibodies are used alone to kill or ablate cancer cells, such killing or ablation can be effected by initiating endogenous host immune functions, such as complement-mediated or antibody-dependent cellular cytotoxicity.

The route of antibody administration is in accord with known methods, e.g., injection or infusion by intravenous, intraperitoneal, intracerebral, intramuscular, subcutaneous, intraocular, intraarterial, intrathecal, inhalation or intralesional routes, or by sustained release systems. The antibody is preferably administered continuously by infusion or by bolus injection. One may administer the antibodies in a local or systemic manner.

The present antibodies may be prepared in a mixture with a pharmaceutically acceptable carrier. Techniques for formulation and administration of the compounds of the instant application may be found in “Remington's Pharmaceutical Sciences,” Mack Publishing Co., Easton, Pa., latest edition. This therapeutic composition can be administered intravenously or through the nose or lung, preferably as a liquid or powder aerosol (lyophilized). The composition may also be administered parenterally or subcutaneously as desired. When administered systematically, the therapeutic composition should be sterile, pyrogen-free and in a parenterally acceptable solution having due regard for pH, isotonicity, and stability. These conditions are known to those skilled in the art.

Pharmaceutical compositions suitable for use include compositions wherein one or more of the present antibodies are contained in an amount effective to achieve their intended purpose. More specifically, a therapeutically effective amount means an amount of antibody effective to prevent, alleviate or ameliorate symptoms of disease or prolong the survival of the subject being treated. Determination of a therapeutically effective amount is well within the capability of those skilled in the art, especially in light of the detailed disclosure provided herein. Therapeutically effective dosages may be determined by using in vitro and in vivo methods.

In a further embodiment, recombinant DNA including an insert coding for a heavy chain variable domain and/or for a light chain variable domain of cancer-binding antibodies described hereinbefore are produced. The term DNA includes coding single stranded DNAs, double stranded DNAs consisting of said coding DNAs and of complementary DNAs thereto, or these complementary (single stranded) DNAs themselves.

Furthermore, DNA encoding a heavy chain variable domain and/or a light chain variable domain of the cancer-binding antibodies disclosed herein can be enzymatically or chemically synthesized DNA having the authentic DNA sequence coding for a heavy chain variable domain and/or for the light chain variable domain, or a mutant thereof. A mutant of the authentic DNA is a DNA encoding a heavy chain variable domain and/or a light chain variable domain of the above-mentioned antibodies in which one or more amino acids are deleted or exchanged with one or more other amino acids. Preferably said modification(s) are outside the CDRs of the heavy chain variable domain and/or of the light chain variable domain of the antibody in humanization and expression optimization applications. The term mutant DNA also embraces silent mutants wherein one or more nucleotides are replaced by other nucleotides with the new codons coding for the same amino acid(s). The term mutant sequence also includes a degenerated sequence. Degenerated sequences are degenerated within the meaning of the genetic code in that an unlimited number of nucleotides are replaced by other nucleotides without resulting in a change of the amino acid sequence originally encoded. Such degenerated sequences may be useful due to their different restriction sites and/or frequency of particular codons which are preferred by the specific host, particularly E. coli, to obtain an optimal expression of the heavy chain murine variable domain and/or a light chain murine variable domain.

The term mutant is intended to include a DNA mutant obtained by in vitro mutagenesis of the authentic DNA according to methods known in the art.

For the assembly of complete tetrameric immunoglobulin molecules and the expression of chimeric antibodies, the recombinant DNA inserts coding for heavy and light chain variable domains are fused with the corresponding DNAs coding for heavy and light chain constant domains, then transferred into appropriate host cells, for example after incorporation into hybrid vectors.

Recombinant DNAs including an insert coding for a heavy chain murine variable domain of an antibody directed to the cell line disclosed herein fused to a human IGg heavy chain constant domain, for example γ1, γ2, γ3 or γ4, preferably γ1 or γ4 are also provided. Recombinant DNAs including an insert coding for a light chain murine variable domain of an antibody directed to the cell line disclosed herein fused to a human constant domain κ or λ, preferably κ are also provided

Another embodiment pertains to recombinant DNAs coding for a recombinant polypeptide wherein the heavy chain variable domain and the light chain variable domain are linked by way of a spacer group, optionally including a signal sequence facilitating the processing of the antibody in the host cell and/or a DNA coding for a peptide facilitating the purification of the antibody and/or a cleavage site and/or a peptide spacer and/or an effector molecule.

The DNA coding for an effector molecule is intended to be a DNA coding for the effector molecules useful in diagnostic or therapeutic applications. Thus, effector molecules which are toxins or enzymes, especially enzymes capable of catalyzing the activation of prodrugs, are particularly indicated. The DNA encoding such an effector molecule has the sequence of a naturally occurring enzyme or toxin encoding DNA, or a mutant thereof, and can be prepared by methods well known in the art.

In order that those skilled in the art may be better able to practice the compositions and methods described herein, the following examples are given for illustration purposes.

### Example 1

Anti-sera from mice immunized with a variety of cancer cell lines were shown to cross-react with human red blood cells (RBC). Seven different cancer cell lines were used to immunize 31 Balb/c mice (see, Table 1). The immune response of these mice was tested against the original cancer cell lines after four rounds of immunization. Pre-bleed serum before immunization and post-bleed serum after immunization were tested against the original cancer cell line by fluorescence-activated cell sorting (FACS). As shown in Table 1, all mice were found to produce a very strong immune response to injected cancer cells. When the same anticancer sera were tested against human RBC, all of the samples were found to cross-react with human RBC. In this study, mice were found to produce a significant amount of antibodies against human RBC after cancer cell immunization, due to the fact that cancer cells and human blood cells share common antigens on the cell surface. Therefore, antibodies that target common antigens on both cancer and RBC cells could interfere with screening of cancer therapeutic antibodies by whole cell panning.

TABLE 1
FACS Analyses of Cross-reactivity of
Anticancer Sera to Human Blood Cells

| | | | FACS with | |
|---|---|---|---|---|
| | | | Original Cancer | FACS with |
| | | | Cells | RBC |
| | | | (Post-bleed/ | (Post-bleed/ |
| | Cancer | Animal | Pre-bleed) | Pre-bleed) |
| Cell Lines | Type | Number | Geo-Mean | Geo-Mean |
| MDA-MB-435 | Breast | 5 | 350 X | 149 X |
| MCF-7 | Breast | 5 | 300 X | 329 X |
| SK-OV3 | Ovarian | 5 | 178 X | 423 X |
| PC3 | Prostate | 4 | 400 X | 516 X |
| Du145 | Prostate | 5 | 420 X | 661 X |
| KM12L4a | Colon | 4 | 300 X | 307 X |
| A431 | Head and | 3 | 275 X | 557 X |
| | Neck | | | |
| Caki-1 | Renal | 3 | 300 X | 160 X |

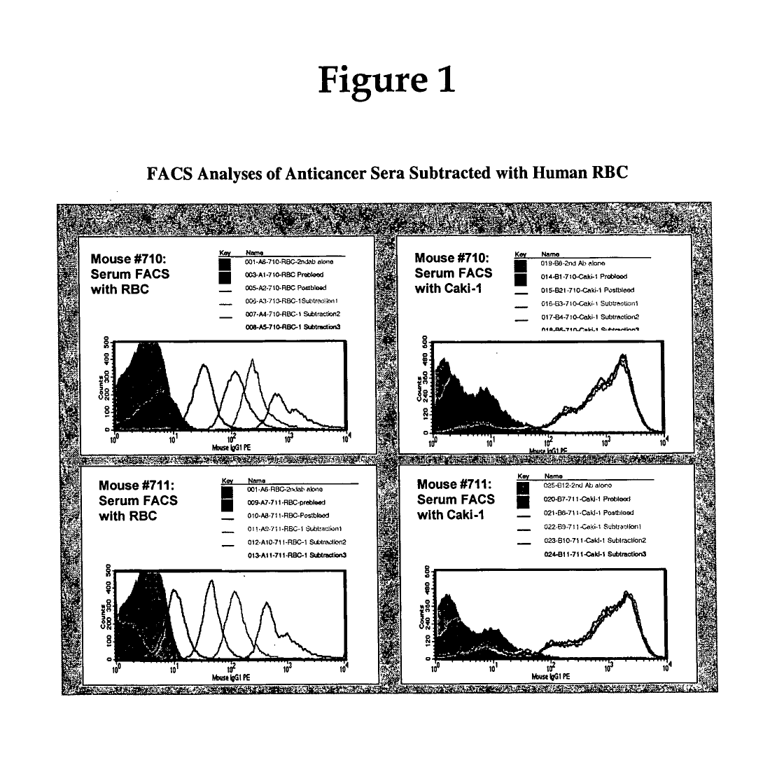

Anti-sera from mice immunized with cancer cells were subjected to RBC subtraction for a total of three times. The remaining antibodies after this subtraction dramatically lost binding activity to RBC but unchanged binding activity to cancer cells (see, FIG. 1), indicating that there was a large population of antibodies against common antigens on both RBC and cancer cells, which could interfere screening of cancer therapeutic antibodies. Thus, non-tumor specific antibodies can be removed by subtraction, which is an arbitrary environment created for antibodies binding to normal cells during whole cell panning. Whole cell panning of the large population of antibodies left after subtraction (which did not bind to RBC) allows selection for tumor specific antibodies.

Balb/c mice were immunized with Renal Cell Carcinoma (“RCC”) cell line Caki-1. Caki-1 (ATCC, HTB-46) is a clear cell renal carcinoma cell line. To perform the first round of RBC subtraction, 500 μl of each post-bleed serum (1:10 dilution) from each mouse was incubated with 5×10⁸ normal human red blood cells type-A (in 100 μl of 1×PBS 1% BSA) at 4° C. for 1 hour with gentle shaking. The red blood cells were spun down at 1800 rpm for 1 minute in a microfuge. The supernatant was kept for FACS analysis and for the next round of subtraction. The second and third rounds of subtraction were performed in this same manner. FACS analysis was performed on serum from each subtraction with red blood cells and Caki-1 tumor cells. For comparison, FACS analysis was performed on pre-bleed serum and post-bleed serum without subtraction.

The polyclonal antibodies remaining after the negative selection process can be used as a therapeutic in treating cancer patients.

### Example 2

### PC3 Cell Panning with and without the Stringent RBC Subtraction

In order to decrease the percentage of antibodies that cross-react with normal cells, especially with human blood cells, whole cell panning was performed with stringent RBC/normal cell subtractions. Normal cells used in whole cell panning were prostate epithelial cells, PrEC (available from Clonetics, San Diego, Calif.). The final antibodies from panning with RBC subtraction were compared with those from similar panning without RBC subtraction.

Mice were immunized with prostate cancer cells known as PC3 cells (ATCC, CRL-1435). There are several advantages to using PC3 cell lines. First, the PC3 cell line is an adenocarcinoma line, which could be used to mimic adenocarcinoma (95% of prostate cancer) in vivo. Second, this cell line is hormone independent, which could be used to mimic the disease population with hormone refractory prostate cancer (“HRPC”). A third advantage is that PC3 cells have a very aggressive tumor growth phenotype. This cell line is metastatic in rodent animal models. In addition, PC3 cells also grow very fast in vitro and are easily manipulated in a cell panning setting.

### Generation of Library DNA

Total RNA was isolated from mouse spleen samples and messenger RNA was purified using Oligotex RNA purification kit (QIAGEN Inc., Valencia, Calif.). First strand cDNA was synthesized using SuperScript II RTase first strand cDNA synthesis kit (Invitrogen Corp., Carlsbad, Calif.). Second strand cDNA synthesis and the amplification of IgG1 and IgG2a heavy chain and the kappa light chain fragments were performed according to the method described in U.S. application Ser. No. 10/251,085, the disclosure of which is incorporated herein in its entirety by this reference. The amplified fragments were purified and digested with appropriate restriction endonucleases and inserted into Fab expression vectors PAX243mG1K for IgG1 kappa library and PAX243mG2aK for IgG2a kappa library.

E. coli Strain Used for Transformation

The library construction was performed by electroporating TOP10F′ cells and/or XL-1 blue cells. The library DNA was purified from overnight culture of E. coli cells using Hi-Speed maxi preparation kit (QIAGEN Inc.).

### IPTG Induction of Phage Amplification

For the panning, the library DNA was electroporated into ER2738 cells and phage production was induced with the addition of VCSM13 helper phage and 1 mM IPTG at 30° C. overnight.

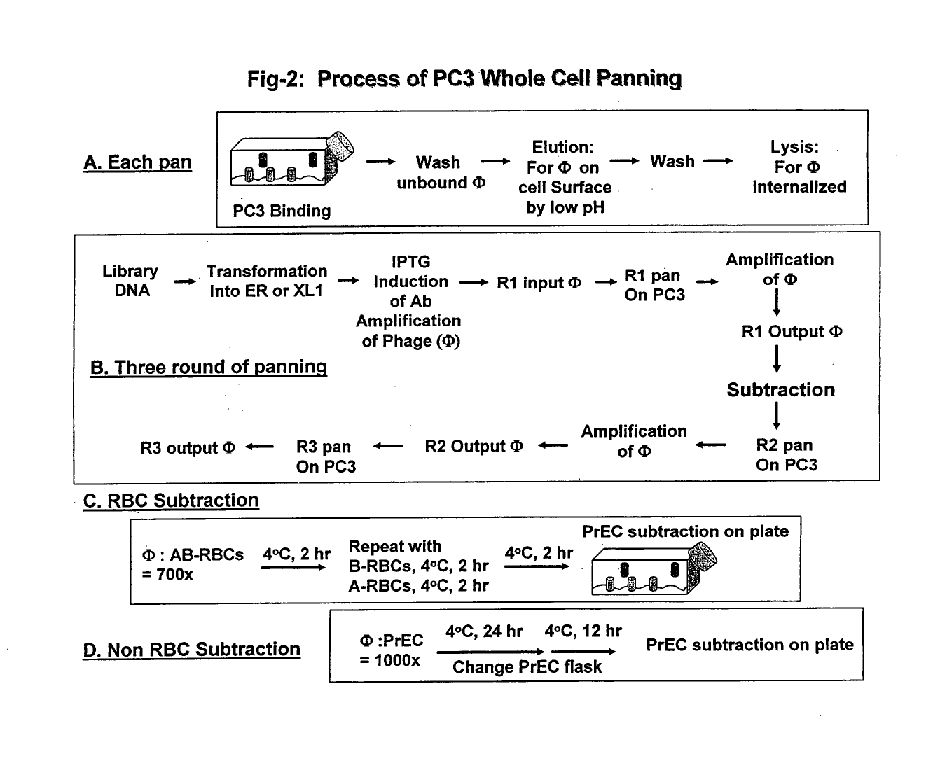

Whole cell panning is used to select antibodies having the desired characteristics. The panning process is schematically summarized in FIG. 2. Expression ELISA is performed to identify Fab-expressing clones. Once Fab-expressing clones are identified, a cell ELISA is performed to identify clones that bind PC3 cells.

After panning on PC3 cells for positive selection, two experiments using different negative selection processes were performed. In the first, subtraction panning was done only with prostate epithelial cells. All the clones from this panning were red blood cell positive clones. The output clones from the round 2 pan and the round 3 pan were screened with antibody expression ELISA, RBC-FACS, PC3 cell ELISA and PrEC cell ELISA. The clones from prostate epithelial cell panning with a phenotype of PC3(+)/PrEC(−) were chosen for DNA sequencing.

In a second experiment, subtraction panning with both prostate epithelial cells and red blood cells was performed. The red blood cell subtraction panning was done in three steps. First, a total of 3.8×10¹² phage from R1 output were mixed with 5.4×10⁹ of type AB red blood cells at a ratio of 700 phage per cell, and incubated at 4° C. for 2 hours. Then, unbound phage were incubated with 5.4×10⁹ of type A red blood cells at 4° C. for 2 hours. Finally, unbound phage were incubated with 5.4×10⁹ of type B red blood cells at 4° C. for another 2 hours. This stringent red blood cell subtraction subtracts out the majority of antibodies that bind to antigens common to both cancer cells and red blood cells. The output clones from the round 2 pan and the round 3 pan were screened with antibody expression ELISA, RBC-FACS, PC3 cell ELISA and PrEC cell ELISA. The clones from the combination whole cell panning (i.e., positive panning on PC3 cells and negative panning on prostate epithelial cells and red blood cells) with a phenotype of PC3(+)/RBC(−)/PrEC(−) were chosen for DNA sequencing. The PC3 cell ELISA data is also validated using PC3 cell FACS.

### In Vitro Antibody Validation

Purified mouse Fab from bacterial lysates are obtained using an anti-Fab column. Immunohistochemistry (IHC) is also performed. Prostate tumor arrays are used to evaluate the binding pattern of each Fab to tumor cells. Normal tissue arrays are used to evaluate the binding of each Fab to normal cells.

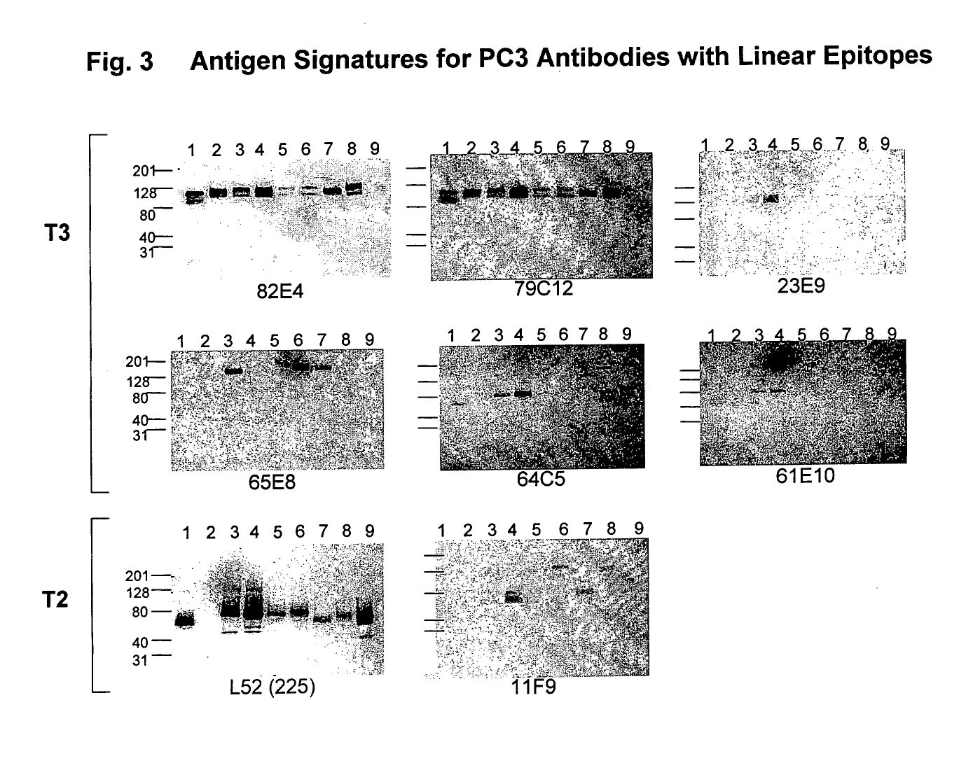

Western blot analysis of antigen signatures is conducted as follows: Total cell lysates from a panel of 9 cell lines are run on non-reducing SDS-PAGE. The loading order and specific cell lines employed are shown in Table 2, below. Each Fab is then used as a primary antibody to determine the molecular weight of an antigen identified in each cell line. Every unique Fab that recognizes a linear epitope will display a distinct pattern of antigen binding in every cell line, resulting in an “antigen signature” (see FIG. 3). Those Fabs that do not recognize a linear epitope do not display an antigen signature, but can still immunoprecipitate antigens. The number of antigens from each pan is compared by antigen signature. Immunoprecipitation and mass spectrometric analysis are used for antigen identification. Specifically, each Fab is used to immunoprecipitate its antigen from PC3 cell membrane preparations. The antigen band is excised from the SDS-PAGE gel (reducing) and sent to the Harvard Microchemistry facility (Cambridge, Mass.) for mass spectrometric analysis and identification of the antigen by peptide digestion/mapping.

TABLE 2

| Loading | | | |
|---|---|---|---|
| Order | Cell Line | Type | Source |
| 1 | Du145 | Prostate carcinoma | ATCC, HTB-81 |
| 2 | PrEC | Normal prostate | Clonetics |
| 3 | PC3 | Prostate | ATCC, CRL-1435 |
| | | adenocarcinoma | |
| 4 | Hela | Cervical carcinoma | ATCC, CCL-2 |
| 5 | MDA-435 | Breast ductal | NCI |
| | | carcinoma | |
| 6 | KM12 | Colon carcinoma | MD-Anderson |
| 7 | SK-OV3 | Ovarian carcinoma | ATCC, HTB-77 |
| 8 | A431 | Squamous | ATCC, CRL-1555 |
| | | epidermoid | |
| | | carcinoma | |
| 9 | A375 | Skin melanoma | ATCC, CRL-1619 |

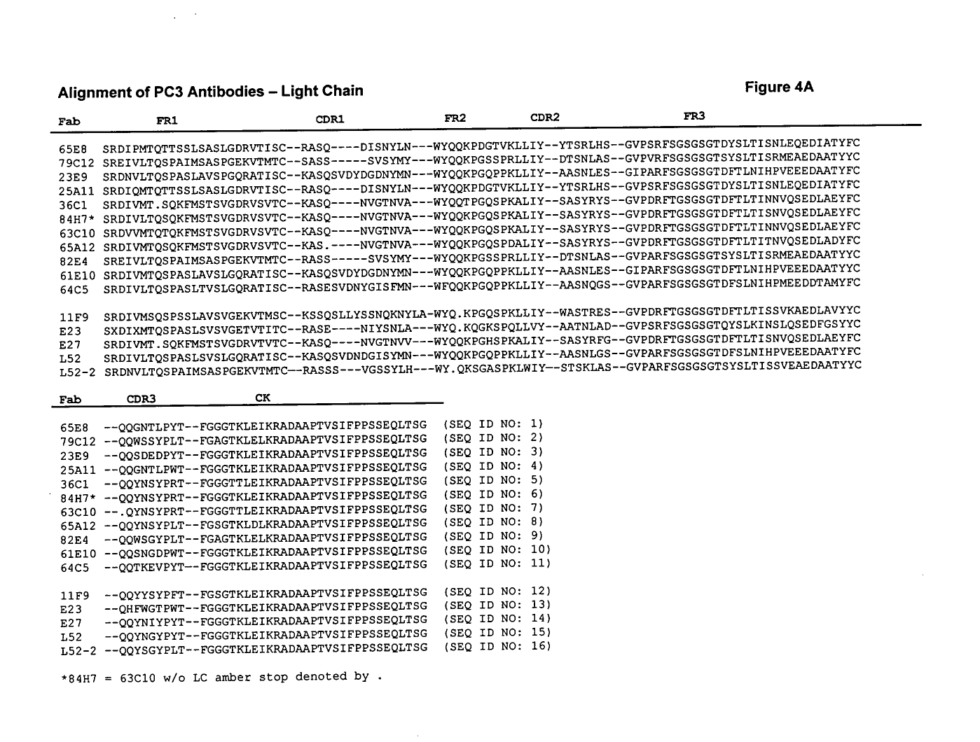

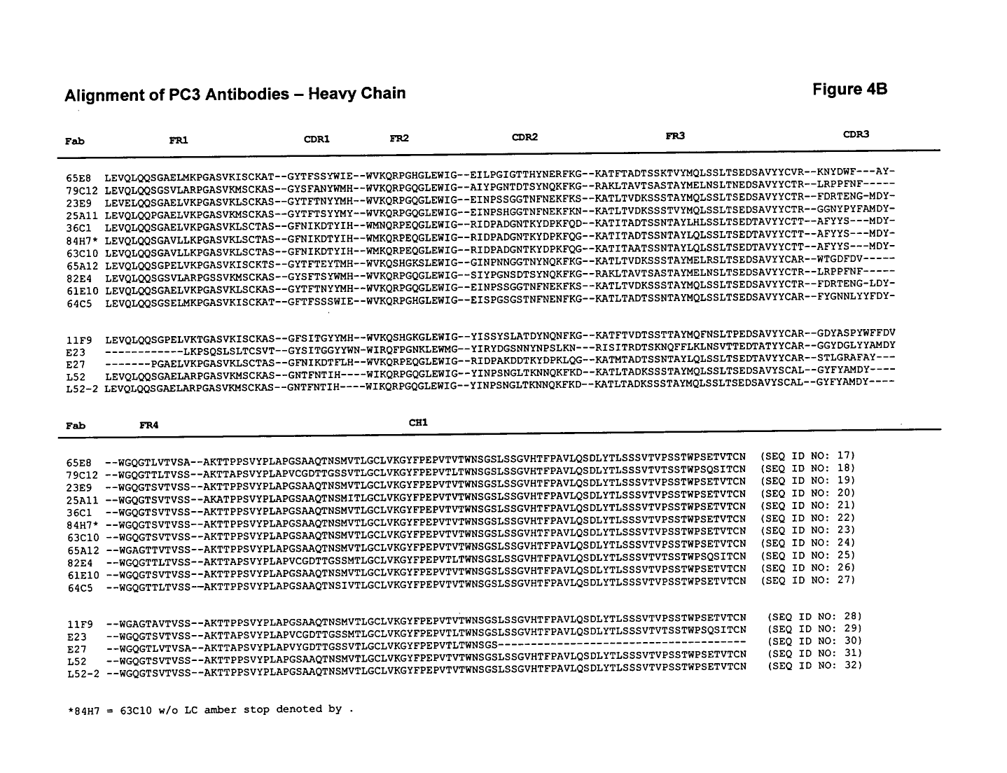

Without red blood cell subtraction, 21 clones were obtained that bind to PC3 cancer cells from a total of 1536 output clones after three rounds of whole cell panning. Of 21 clones, all were found to bind red blood cells. Sequences of 21 clones were clustered into four major groups, L52-2 group, E23 group, 11F9 group and E27 group (FIGS. 4A and 4B). 11F9 bids to a protein with a molecular weight of 20 Kd. The sequences of clones in each group only have a couple of amino acid differences. Fab L52-2 and E23 bind to CD55, which is highly expressed on red blood cells and PC3 cells, but not on PrEC cells. Fab E27 binds to an unknown antigen. These results suggested that PC3 cells express significant amount of common antigens to red blood cells, which interferes with positive selection.

Using red blood cell subtraction, 146 clones were obtained that bind to PC3 cancer cells from a total of 4416 output clones after three rounds of whole cell panning (R3). Of 146 clones, 24 were found not to bind red blood cells. These 24 clones were sequenced. With the addition of two clones from R2 pan, a total of 10 clones were obtained with different Fab sequences (FIGS. 4A and 4B). The final 10 clones bind to PC3 cancer cells, but do not bind to human red blood cells. Of these 10 clones, five do not bind PrEC. In comparison with antibodies from the whole cell panning without red blood cell subtraction, stringent subtraction indeed increased antibody diversity and yielded a desirable profiles, which is PC3⁽⁺⁾/PrEC^{(-/+)}/RBC⁽⁻⁾.

TABLE 3
PC3 antigens identified by immunoprecipitation
and mass spectrometry.

| Antibody | Antigen |
|---|---|
| 65E8 | CD26 (DPPIV) |
| 79C12 | Integrin alpha2/beta1 |
| 23E9 | Integrin alpha3/beta1 |
| 25A11 | Cdcp1 |
| 36C1 | Integrin alpha3/beta1 |
| 84H7 (63C10) | Integrin alpha3/beta1 |
| 65A12 | Integrin beta4 |
| 82E4 | Integrin alpha2/alpha3/alpha5/beta1 |
| 61E10 | Integrin alpha3/beta1 |
| 64C5 | Unknown |
| 11F9 | Unknown (P20) |
| E23 | CD55 |
| E27 | Unknown |
| L52 | CD55 |

It will be understood that various modifications may be made to the embodiments disclosed herein. For example, as those skilled in the art will appreciate, the specific sequences described herein can be altered slightly without necessarily adversely affecting the functionality of the antibody or antibody fragment. For instance, substitutions of single or multiple amino acids in the antibody sequence can frequently be made without destroying the functionality of the antibody or fragment. Thus, it should be understood that antibodies having a degree of identity greater than 70% to the specific antibodies described herein are within the scope of this disclosure. In particularly useful embodiments, antibodies having an identity greater than about 80% to the specific antibodies described herein are contemplated. In other useful embodiments, antibodies having an identity greater than about 90% to the specific antibodies described herein are contemplated. Therefore, the above description should not be construed as limiting, but merely as exemplifications of preferred embodiments. Those skilled in the art will envision other modifications within the scope and spirit of the present disclosure.

1. A method comprising:
collecting antiserum from subjects immunized with a cancer cell;
contacting the antiserum with human red blood cells; and
recovering the portion of the antiserum that does not bind to the human red blood cells.

2. The method of claim 1 further comprising the steps of contacting the antiserum antibodies that bind to human white blood cells and recovering the portion of the antiserum that does not bind to the human white blood cells.

3. The method of claim 1 further comprising the step of contacting the antiserum antibodies that bind to human non-cancerous cells and recovering the portion of the antiserum that does not bind to the human non-cancerous cells.

4. A method comprising:
collecting antiserum from subjects immunized with a cancer cell;
removing from the antiserum antibodies that bind to human red blood cells; and
recovering from the antiserum antibodies that bind to the cancer cell.

5. The method of claim 4 further comprising the step of removing from the antiserum antibodies that bind to human white blood cells.

6. The method of claim 4 further comprising the step of removing from the antiserum antibodies that bind to human non-cancerous cells.

7. A method comprising:
a) collecting antiserum from subjects immunized with a cancer cell;
b) removing from the antiserum
i) antibodies that bind to human red blood cells and
ii) antibodies that bind to at least one other type of non-cancerous cell selected from the group consisting of endothelial cells, epithelial cells, smooth muscle cells, liver cells, lung cells, heart cells, kidney cells, intestine cells, stomach cells, bladder cells, spleen cells, pancreas cells, bone marrow cells, brain cells, thymus cells, prostate cells, ovary cells, testis cells and skin cells; and
c) then recovering from the antiserum antibodies that bind to the cancer cell.

8. The method of claim 7 further comprising the step of removing from the antiserum antibodies that bind to human white blood cells.

9. A method comprising:
a) collecting antiserum from subjects immunized with a cancer cell;
b) mixing human red blood cells with the antiserum;
c) removing the human red blood cells and antibodies bound thereto from the mixture and recovering a first portion of the antiserum;
d) mixing human red blood cells with the first portion of the antiserum;
e) removing the human red blood cells and antibodies bound thereto from the mixture and recovering a second portion of the antiserum;
f) mixing human red blood cells with the second portion of the antiserum;
g) removing the human red blood cells and antibodies bound thereto from the mixture and recovering a third portion of the antiserum; and
h) recovering from the third portion of the antiserum antibodies that bind to the cancer cell.

10. The method of claim 9 further comprising the steps
i) mixing human white blood cells with the third portion of the antiserum;
j) removing the human white blood cells and antibodies bound thereto from the mixture and recovering a fourth portion of the antiserum; and
k) recovering from the fourth portion of the antiserum antibodies that bind to the cancer cell.

11. A method comprising:
a) generating a phage displayed antibody library using cells collected from subjects immunized with cancer cells;
b) removing members of the library that bind to human red blood cells to generate a sub-library; and
c) recovering from the sub-library members that display antibodies that bind to the cancer cell.

12. The method of claim 11 further comprising the step of removing members of the library that bind to human white blood cells.

13. The method of claim 11 further comprising the step of removing members of the library that bind to normal tissue cells.

14. An antibody that binds to a prostate cancer cell comprising a light chain CDR1 selected from the group consisting of RASQDISNYLN (SEQ ID NO: 33), SASSSVSYMY (SEQ ID NO: 34), KASQSVDYDGDNYMN (SEQ ID NO: 35), KASQNVGTNVA (SEQ ID NO: 36), RASSSVSYMY (SEQ ID NO: 37), RASESVDNYGISFMN (SEQ ID NO: 38), KSSQSLLYSSNQKNYLA (SEQ ID NO: 39), RASENIYSNLA (SEQ ID NO: 40), KASQNVGTNVV (SEQ ID NO: 41), KASQSVDNDGISYMN (SEQ ID NO: 42), and RASSSVGSSYLH (SEQ ID NO: 43).

15. An antibody that binds to a prostate cancer cell comprising a light chain CDR2 selected from the group consisting of YTSRILHS (SEQ ID NO: 44), DTSNLAS (SEQ ID NO: 45), AASNLES (SEQ ID NO: 46), SASYRYS (SEQ ID NO: 47), AASNQGS (SEQ ID NO: 48), WASTRES (SEQ ID NO: 49), AATNLAD (SEQ ID NO: 50), SASYRFG (SEQ ID NO: 51), AASNLGS (SEQ ID NO: 52), and STSKLAS (SEQ ID NO: 53).

16. An antibody that binds to a prostate cancer cell comprising a light chain CDR3 selected from the group consisting of QQGNTLPYT (SEQ ID NO: 54), QQWSSYPLT (SEQ ID NO: 55), QQSDEDPYT (SEQ ID NO: 56), QQGNTLPWT (SEQ ID NO: 57), QQYNSYPRT (SEQ ID NO: 58), QQYNSYPLT (SEQ ID NO: 59), QQWSGYPLT (SEQ ID NO: 60), QQSNGDPWT (SEQ ID NO: 61), QQTKEVPYT (SEQ ID NO: 62), QQYYSYPFT (SEQ ID NO: 63), QHFWGTPWT (SEQ ID NO: 64), QQYNIYPYT (SEQ ID NO: 65), QQYNGYPYT (SEQ ID NO: 66), and QQYSGYPLT (SEQ ID NO: 67).

17. An antibody that binds to a prostate cancer cell comprising a heavy chain CDR1 selected from the group consisting of GYTFSSYWIE (SEQ ID NO: 68), GYSFANYWMH (SEQ ID NO: 69), GYTFTNYYMH (SEQ ID NO: 70), GYTFTSYYMY (SEQ ID NO: 71), GFNIKDTYIH (SEQ ID NO: 72), GYTFTEYTMH (SEQ ID NO: 73), GYSFTSYWMH (SEQ ID NO: 74), GFTFSSSWIE (SEQ ID NO: 75), GFSITGYYMH (SEQ ID NO: 76), GYSITGGYYWN (SEQ ID NO: 77), GFNIKDTFLH (SEQ ID NO: 78), and GNTFNTIH (SEQ ID NO: 79).

18. An antibody that binds to a prostate cancer cell comprising a heavy chain CDR2 selected from the group consisting of EILPGIGTTHYNERFKG (SEQ ID NO: 80), AIYPGNTDTSYNQKFKG (SEQ ID NO: 81), EINPSSGGTNFNEKFKS (SEQ ID NO: 82), EINPSHGGTNFNEKFKN (SEQ ID NO: 83), RIDPADGNTKYDPKFQD (SEQ ID NO: 84), RIDPADGNTKYDPKFQG (SEQ ID NO: 85), GINPNNGGTNYNQKFKG (SEQ ID NO: 86), SIYPGNSDTSYNQKFKG (SEQ ID NO: 87), EISPGSGSTNFNENFKG (SEQ ID NO: 88), YISSYSLATDYNQNFKG (SEQ ID NO: 89), YIRYDGSNNYNPSLKN (SEQ ID NO: 90), RIDPAKDDTKYDPKLQG (SEQ ID NO: 91), and YINPSNGLTKNNQKFKD (SEQ ID NO: 92).

19. An antibody that binds to a prostate cancer cell comprising a heavy chain CDR3 selected from the group consisting of KNYDWFAY (SEQ ID NO: 93), LRPPFNF (SEQ ID NO: 94), FDRTENGMDY (SEQ ID NO: 95), GGNYPYFAMDY (SEQ ID NO: 96), AFYYSMDY (SEQ ID NO: 97), WTGDFDV (SEQ ID NO: 98), FDRTENGLDY (SEQ ID NO: 99), FYGNNLYYFDY (SEQ ID NO: 100), GDYASPYWFFDV (SEQ ID NO: 101), GGYDGLYYAMDY (SEQ ID NO: 102), STLGRAFAY (SEQ ID NO: 103), and GYFYAMDY (SEQ ID NO: 104).

20. An antibody that binds to a prostate cancer cell comprising an amino acid sequence selected from the group consisting of SEQ ID NOS: 1-16.

21. An isolated nucleic acid encoding an antibody of claim 14.

22. An expression vector comprising an isolated nucleic acid in accordance with claim 21.

23. A host cell transfected with an expression vector in accordance with claim 22.

24. A method comprising:
a) contacting cancer cells with a hapten;
b) generating a phage displayed antibody library using cells collected from subjects immunized with the cancer cells;
c) removing members of the library that bind to human red blood cells to generate a sub-library; and
d) recovering from the sub-library members that display antibodies that bind to the cancer cell.

25. The method of claim 24 wherein the hapten is dinitrophenyl.

26. The method of claim 24 further comprising the step of removing members of the library that bind to human white blood cells.

27. The method of claim 24 further comprising the step of removing members of the library that bind to normal tissue cells.

28. An antibody that binds to Cdcp1 comprising an amino acid sequence of selected from the group consisting of SEQ ID NO: 4, SEQ ID NO: 20, SEQ ID NO: 33, SEQ ID NO: 44, SEQ ID NO: 57, SEQ ID NO: 71, SEQ ID NO: 83 and SEQ ID NO: 96.

29. An isolated nucleic acid encoding an antibody of claim 15.

30. An expression vector comprising an isolated nucleic acid in accordance with claim 29.

31. A host cell transfected with an expression vector in accordance with claim 30.

32. An isolated nucleic acid encoding an antibody of claim 16.

33. An expression vector comprising an isolated nucleic acid in accordance with claim 32.

34. A host cell transfected with an expression vector in accordance with claim 33.

35. An isolated nucleic acid encoding an antibody of claim 17.

36. An expression vector comprising an isolated nucleic acid in accordance with claim 35.

37. A host cell transfected with an expression vector in accordance with claim 36.

38. An isolated nucleic acid encoding an antibody of claim 18.

39. An expression vector comprising an isolated nucleic acid in accordance with claim 38.

40. A host cell transfected with an expression vector in accordance with claim 39.

41. An isolated nucleic acid encoding an antibody of claim 19.

42. An expression vector comprising an isolated nucleic acid in accordance with claim 41.

43. A host cell transfected with an expression vector in accordance with claim 42.

44. An isolated nucleic acid encoding an antibody of claim 20.

45. An expression vector comprising an isolated nucleic acid in accordance with claim 44.

46. A host cell transfected with an expression vector in accordance with claim 45.
